(19) United States
(12) Patent Application Publication
(10) Pub. No.: US 2014/0302013 A1
(43) Pub. Date: Oct. 9, 2014

(54) PREDICTING AND DIAGNOSING PATIENTS WITH SYSTEMIC LUPUS ERYTHEMATOSUS

(71) Applicant: OKLAHOMA MEDICAL RESEARCH FOUNDATION, Oklahoma City, OK (US)

(72) Inventors: Christopher J. Lessard, Oklahoma City, OK (US); Kathy L. Sivils, Oklahoma City, OK (US)

(73) Assignee: OKLAHOMA MEDICAL RESEARCH FOUNDATION, Oklahoma City, OK (US)

(21) Appl. No.: 14/248,038

(22) Filed: Apr. 8, 2014

Related U.S. Application Data
(60) Provisional application No. 61/809,675, filed on Apr. 8, 2013.

(51) Int. Cl. C12Q 1/68

(52) U.S. Cl. CPC C12Q 1/6827
USPC 424/133.1; 435/6.11

(57) ABSTRACT

The present invention provides methods for the prediction and diagnosis of Systemic Lupus Erythematosus using single nucleotide polymorphisms in IRF8.

This application claims benefit of priority to U.S. Provisional Application Ser. No. 61/809,675, filed Apr. 8, 2013, the entire contents of which are hereby incorporated by reference.

This invention was made with Government support under N01 AR62277 and R01 AR043274 awarded by the National Institutes of Health. The Government has certain rights in the invention.

### BACKGROUND OF THE INVENTION

A. Field of the Invention

The present invention relates to the fields of molecular biology, pathology and genetics. More specifically, the invention relates to methods of predicting and diagnosing automimmune disease based on the presence or absence of single nucleotide polymorphisms.

B. Related Art

Autoimmune diseases comprise a large number of widely varying illnesses. Their common feature is the existence of an immune response in the subject against one or more “self” antigens, including such wide ranging molecules as proteins, DNA and carbohydrates. These diseases can cause symptoms ranging from only mild discomfort to the patient, to complete debilitation and death. Most of autoimmune diseases remain very enigmatic, not only in their molecular basis and precipitating factors, but in their prediction, progression and treatment. As such, they continue to provide a considerable challenge to the healthcare industry.

Most genetic-based diseases do not generally have a simple, single genetic cause. Moreover, they are usually affected by environmental factors as well. The same can be said for autoimmune diseases, where defects in multiple genes often are involved. The situation is not aided by clinical diagnosis, since (a) familial autoimmune disease is often characterized by related individuals suffering from distinct autoimmune defects, and (b) the same autoimmune disease may manifest itself differently in different individuals at different times. Thus, one is left with a difficult, if not impossible, clinical diagnosis even when some genetic information is available. That is why researches continue to seek out better and more complete genetic bases for autoimmune diseases.

Systemic Lupus Erythematosus (SLE), like other autoimmune diseases, is mediated by a complex interaction of genetic and environmental elements. The genetic component of this interaction is clearly important: 20% of people with SLE have a relative who has or will have SLE. It is commonly believed that environmental factors may trigger a genetic predisposition to such diseases. Although the crucial role of genetic predisposition in susceptibility to SLE has been known for decades, only minimal progress has been made towards elucidating the specific genes involved in human disease. It is also suspected that SLE may be related to genetic defects in apoptosis. For example, mice lacking the gene for DNase1 develop SLE by 6 to 8 months of age.

Family studies have identified a number of genetic regions associated with elevated risk for SLE, although no specific genes have yet been identified (Harley et al., 1998; Wakeland et al., 2001). For example, 1q42 has been linked to SLE in three independent studies (reviewed in Gaffney et al., 1998). Other genetic locations revealed by model-based linkage analysis include 1q23 and 11q14 in African Americans, 14q11, 4p15, 11q25, 2q32, 19q13, 6q26-27, and 12p12-11 in European Americans, with 1q23, 13q32, 20q13, and 1q31 showing up in combined pedigrees (Moser et al., 1998). Associations have also been shown for the genetic markers HLA-DR2 and HLA-DR3 (Arnett et al., 1992). More recently, expression profiling of peripheral blood mononuclear cells of SLE patients using microarrays has shown that about half of the patients demonstrate disregulated expression of genes in the IFN pathway (Baechler et al., 2003).

Despite these important observations, it is far from clear that one can predict the existence or predisposition to SLE based on this handful of genetic information. In all likelihood, a much more robust analysis using more and better genetic markers to identify SLE (and distinguish it from other autoimmune diseases) will be required.

### SUMMARY OF THE INVENTION

Thus, in accordance with the present invention, there is provided a method of identifying a subject afflicted with or at risk of developing Systemic Lupus Erythematosus comprising (a) providing a nucleic acid-containing sample from the subject; (b) determining the presence or absence of a single nucleotide polymorphism (SNP) in IRF8; and (c) identifying said subject as afflicted or at risk of development of Systemic Lupus Erythematosus when the presence of a SNP in IRF8 is observed. The method may further comprise determining the presence or absence of a second SNP from IRF8. The SNP)s) is/are rs11644034 and/or rs11648084.

The method may further comprise treating the subject based on the results of step (b). The method may also further comprise taking a clinical history from the subject. Determining may comprise nucleic acid amplification, such as PCR. Determining may comprise primer extension, restriction digestion, sequencing, SNP specific oligonucleotide hybridization and/or DNAse protection assay. The sample may be blood, sputum, saliva, mucosal scraping or tissue biopsy. Determining may comprise assessing the presence or absence of a genetic marker that is in linkage disequilibrium with one or more of rs11644034 and/or 11648084. The method may further comprise obtaining the sample from the subject.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawings will be provided by the Office upon request and payment of the necessary fee.

FIGS. 1A-D. Variants in the region of IRF8 tested for association with SLE. Association of IRF8 with SLE in European (FIG. 1A), African-American (FIG. 1B), and Asian (FIG. 1C) ancestral populations are given with observed (blue diamonds) and imputed (red circles) variants. The dotted line represents the Bonferroni-corrected threshold for the fine-mapping study, with P=1.09×10⁻⁴. The solid black line represents the recombination rate. The variants labeled with blue text represent the most significant observed SNPs, while the one in red represents the most significant SNPs after imputation. In the Asians, rs11117427 was both the most significant observed and imputed variant. (FIG. 1D) Shown is an expanded view of the most statistically significant region in Europeans (circles), African-Americans (triangles), and Asians (squares) for selected variants tagged by rs8046526 (purple), rs450443 (turquoise), rs4843869 (yellow-orange), and rs11117427 (green). Recomb., recombination.

FIG. 2. Conditional analysis results conducted in individuals of European ancestry. The results of the conditional analysis using the 4 SNPs showing peak association in Europeans (rs8046526 and rs4843869), African-Americans (rs450443) and Asians (rs11117427). The black dot represents the non-adjusted single marker association with SLE.

FIGS. 3A-C. IRF8 haplotype and linkage disequilibrium in the European ancestral population. (FIG. 3A) Haplotype structure in Europeans present at a frequency >3%. Major alleles are represented by red squares while the green squares are minor alleles. (FIGS. 3B-D) Linkage disequlibrium plot of r² (FIG. 3B) and D′ (FIG. 3C) in Europeans illustrates the variants tagged by rs450443 and those by rs4843869 are in weak r² but strong D′, providing evidence that these variants are inherited together.

FIGS. 4A-F. Relative expression of IRF8 and a neighboring long non-coding RNA (lncRNA). FIGS. 4A-B show relative expression of IRF8 mRNA by (quantitative polymerase chain reaction (qPCR; normalized to the housekeeping gene HMBS) and IRF8 protein by western blot (normalized to β-actin), respectively, with all data obtained from one experiment. FIGS. 4C and 4E demonstrate relative expression of IRF8 mRNA normalized to HMBS (housekeeping transcript) for IRF8, while FIGS. 4D and 4F demonstrate the relative expression by qPCR of a lncRNA neighboring IRF8. FIGS. 4C-F were generated in two separate experiments. All individuals are stratified as homozygous risk or homozygous non-risk based on the presence of risk or non-risk alleles falling in two haplotype blocks: block 1 (spanning the genomic range falling between SNPs rs9936079 and rs396987) and block 2 (spanning the genomic range falling between SNPs rs4843865 and rs7202472). NS=not significant. Each plot shows error bars with the mean plus or minus the standard error of the mean (SEM).

### DETAILED DESCRIPTION OF THE INVENTION

The interferon regulator factor genes are a family of transcription factors that play a critical role in the regulation of several pathways, including response to pathogens, apoptosis, the cell cycle, and hematopoietic differentiation.⁵⁰ IRF8 is expressed in the nucleus (but partially in the cytoplasm) of B cells, macrophages, and CD11b dendritic cells (DC).⁵⁰ IRF8 can be induced by interferon-γ in macrophages and antigen stimulation within T cells. It also plays an important role in the development of B cells and macrophages.⁵⁰ In the nucleus, IRF8 is required for promoting type I interferon responses in DCs upon viral stimulation.⁵⁰ Interestingly, the overexpression of genes induced by type I interferons has been widely reported in SLE and other autoimmune conditions.^{12,51,52} In the cytosol, IRF8 is involved in the TLR9-MyD88-dependent signaling by binding to TRAF6 in both DCs and macrophages.⁵⁰ After TLR9 stimulation, DCs from mice that are Irf8−/− cannot activate NF-KB or MAPKs.⁵⁰ Of note, rs17445836, which was not included in this study but has been associated with multiple sclerosis (MIM 126200), lies approximately 61 kB telomeric of IRF8, far removed from the regions identified in SLE.⁵³

Fine-mapping, resequencing, imputation, and haplotype analysis of the IRF8 locus in Europeans identified a single haplotype requiring the presence of three independent effects to confer risk. Additionally, several variants within the IRF8 risk haplotype may influence binding to the many regulatory elements present within the region. Thus, the inventors hypothesize that the likely functional effect would result in altered IRF8 mRNA and protein expression. Although the inventors believe that most of the common variation within the region of IRF8 has been evaluated in this study, it is possible that some variants with minor allele frequencies <1% also play a role in SLE risk, but were not detected in this study given the number of samples resequenced.

The TMEM39A associated coding SNP (rs1132200) results in an amino acid change from alanine to threonine at position 487 of the protein. While almost no biological data have been published suggesting its relevance to SLE, it has been found to be associated with multiple sclerosis.⁵⁴ Mechanistic and fine-mapping experiments are needed to better understand if the coding SNP in TMEM39A is functionally relevant or merely correlated with other unexamined causal polymorphism(s).

Though the region surrounding the IKZF3-ZPBP2 locus on 17q21 has been associated with multiple phenotypes, the extensive LD in the region has prohibited investigators from clearly determining the relevant gene. Crohn's disease (MIM 266600), ulcerative colitis (MIM 266600), primary biliary cirrhosis (MIM 109720), and rheumatoid arthritis (MIM 180300) have all reported associations with genes between 34.62-35.51 MB of Chromosome 17.⁵⁵⁻⁶⁰ Fine-mapping and resequencing of this region in Europeans and African-Americans are needed to more precisely refine this association and determine the gene conferring risk. IKZF3 is a member of the IKAROS family of transcription factors involved in lymphocyte development, of which IKZF1 has already been reported as a risk locus for SLE.²² Mice with a mutant form of IKZF3 protein produce anti-dsDNA autoantibodies, making it an interesting candidate gene for human SLE.⁶¹ Moreover, mice that are null for IKZF3 and OBF-1 (POU class 2 associating factor 1) do not mount an autoimmune response.⁶¹ Since the peak signal in this study was in a region containing multiple regulatory elements, it is likely that the associated SNP could affect expression of IKZF3 or ZPBP2, which share the promoter region. However, no known function of ZPBP2 has been reported.

Eleven additional regions were replicated in the European subjects, but did not surpass genome-wide significance. Of these regions, LOC730108/IL12A was previously reported as a risk locus for primary biliary cirrhosis and multiple sclerosis.^{60,62} IL-12A induces interferon-γ and helps differentiate Th1 and Th2 cells.⁶³ The response of lymphocytes to IL-12A is mediated by STAT4, which is also implicated in SLE pathogenesis.⁶⁴ The LIM domain containing preferred translocation partner in lipoma (LPP) is involved in focal adhesions, cell-cell adhesion, and cell motility. Variants within the LPP region have been associated with vitiligo and celiac disease. 65,66 _{To} confirm these associations, replication should be undertaken in a larger independent and equally diverse population.

In conclusion, the inventors have robustly established three additional susceptibility loci for SLE: IRF8, TMEM39A, and IKZF3-ZPBP2. Eleven other regions replicated but did not exceed the genome-wide threshold of significance. Collectively, these data, along with other previously reported loci, demonstrate the growing complexity of the heritable contribution to SLE pathogenesis. A complete understanding of how genetics influence the pathophysiology of SLE will only be possible once the inventors have identified all contributing loci and functional/causal variants for each association, and have extensively evaluated the role of rare variants. More work is needed to increase the understanding of how the loci identified in this study influence SLE etiology.

### II. IRF8

Interferon regulatory factor 8 (IRF8) also known as interferon consensus sequence-binding protein (ICSBP), is a protein that in humans is encoded by the IRF8 gene. IRF8 is a transcription factor that plays critical roles in the regulation of lineage commitment and in myeloid cell maturation including the decision for a common myeloid progenitor (CMP) to differentiate into a monocyte precursor cell.

Interferon Consensus Sequence-binding protein (ICSBP) is a transcription factor of the interferon regulatory factor (IRF) family. Proteins of this family are composed of a conserved DNA-binding domain in the N-terminal region and a divergent C-terminal region that serves as the regulatory domain. The IRF family proteins bind to the IFN-stimulated response element (ISRE) and regulate expression of genes stimulated by type I IFNs, namely IFN-α and IFN-13. IRF family proteins also control expression of IFN-α and IFN-β-regulated genes that are induced by viral infection.

IFN-producing cells (mIPCs) were absent in all lymphoid organs from ICSBP knockout (KO) mice, as revealed by lack of CD11c^{low}B220⁺Ly6C⁺CD11b⁻ cells. In parallel, CD11c⁺ cells isolated from ICSBP KO spleens were unable to produce type I IFNs in response to viral stimulation. ICSBP KO mice also displayed a marked reduction of the DC subset expressing the CD8α marker (CDα⁺ DCs) in spleen, lymph nodes, and thymus. Moreover, ICSBP-deficient CD8α⁺ DCs exhibited a markedly impaired phenotype when compared with WT DCs. They expressed very low levels of costimulatory molecules (intercellular adhesion molecule ICAM1, CD40, CD80, CD86) and of the T cell area-homing chemokine receptor CCR7.

In myeloid cells, IRF8 regulates the expression of Bax and Fas to regulate apoptosis. In chronic myelogenous leukemia (CML), IRF8 regulates acid ceramidaseto mediate CML apoptosis. IRF8 is highly expressed in myeloid cells and was originally identified in as a critical linage-specific transcription factor for myeloid cell differentiation, recent studies, however, have shown that IRF8 is also constitutively expressed in non-hematopoietic cancer cells, albeit at a lower level. Furthermore, IRF8 can also be up-regulated by IFN-γ in non-hemotopoietic cells. IRF8 mediates the expression of Fas, Bax, FLIP, Jak1 and STAT1 to mediate apoptosis in non-hemotopoietic cancer cells.

As discussed below, the present inventors have identified at least two distinct SNPs within the IRF8 gene that have a significant statistical correlation with SLE. The inventors propose that by examining these SNPs, it is possible identify those subjects with SLE, as well as those at risk of developing SLE. The accession number for the human mRNA sequence is NM_{—}002163 and the protein sequence is NP_{—}002154, both of which are incorporated by reference.

### III. SNP-BASED DIAGNOSTICS

Knowledge of DNA polymorphisms can prove very useful in a variety of applications, including diagnosis and treatment of autoimmune disease. A particular kind of polymorphism, called a single nucleotide polymorphism, or SNP (pronounced “snip”), is a small genetic change or variation that can occur within a person's DNA sequence. The genetic code is specified by the four nucleotide “letters” A (adenine), C (cytosine), T (thymine), and G (guanine). SNP variation occurs when a single nucleotide, such as an A, replaces one of the other three nucleotide letters—C, G, or T.

An example of a SNP is the alteration of the DNA segment AAGGTTA to ATGGTTA, where the second “A” in the first snippet is replaced with a “T.” On average, SNPs occur in the human population more than 1 percent of the time. Because only about 3 to 5 percent of a person's DNA sequence codes for the production of proteins, most SNPs are found outside of “coding sequences.” SNPs found within a coding sequence are of particular interest to researchers because they are more likely to alter the biological function of a protein. Because of the recent advances in technology, coupled with the unique ability of these genetic variations to facilitate gene identification, there has been a recent flurry of SNP discovery and detection.

Finding single nucleotide changes in the human genome seems like a daunting prospect, but over the last 20 years, biomedical researchers have developed a number of techniques that make it possible to do just that. Each technique uses a different method to compare selected regions of a DNA sequence obtained from multiple individuals who share a common trait. In each test, the result shows a physical difference in the DNA samples only when a SNP is detected in one individual and not in the other.

Many common diseases in humans are not caused by a genetic variation within a single gene, but instead are influenced by complex interactions among multiple genes as well as environmental and lifestyle factors. Although both environmental and lifestyle factors add tremendously to the uncertainty of developing a disease, it is currently difficult to measure and evaluate their overall effect on a disease process. Therefore, when looking at SNPs, one refers mainly to a person's genetic predisposition, or the potential of an individual to develop a disease based on genes and hereditary factors. This is particularly true in diagnosis of autoimmune disease.

Each person's genetic material contains a unique SNP pattern that is made up of many different genetic variations. Researchers have found that most SNPs are not responsible for a disease state. Instead, they serve as biological markers for pinpointing a disease on the human genome map, because they are usually located near a gene found to be associated with a certain disease. Occasionally, a SNP may actually cause a disease and, therefore, can be used to search for and isolate the disease-causing gene.

To create a genetic test that will screen for an autoimmune disease, one will collect blood or tissue samples from a group of individuals affected by the disease and analyze their DNA for SNP patterns. One then compares these patterns to patterns obtained by analyzing the DNA from a group of individuals unaffected by the disease. This type of comparison, called an “association study,” can detect differences between the SNP patterns of the two groups, thereby indicating which pattern is most likely associated with the disease-causing gene. Eventually, SNP profiles that are characteristic of a variety of diseases will be established. These profiles can then be applied to the population at general, or those deemed to be at particular risk of developing an autoimmune disease.

The examples provide data using Illumina's iSelect HD Custom Genotyping BeadChips® which interrogate virtually any SNP for any species. Custom genotyping panels can be prepared including from 3,072 to 1,000,000 attempted bead types. The BeadChips can be deployed on either the 24-sample (3,072 to 90,000), 12-sample (90,001 to 250,000), or 4-sample (250,001 to 1,000,000) BeadChip format.

However, numerous other approaches to SNP interrogation can be employed, as discussed below. There are a large variety of techniques that can be used to assess SNPs, and more are being discovered each day. The following is a very general discussion of a few of these techniques that can be used in accordance with the present invention.

A. RFLP

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even strains) from one another.

Restriction endonucleases in turn are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in SNP analysis requires that the SNP affect cleavage of at least one restriction enzyme site.

B. Primer Extension

The primer and no more than three NTPs may be combined with a polymerase and the target sequence, which serves as a template for amplification. By using less than all four NTPs, it is possible to omit one or more of the polymorphic nucleotides needed for incorporation at the polymorphic site. It is important for the practice of the present invention that the amplification be designed such that the omitted nucleotide(s) is(are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present. A specific form of primer extension can be found in U.S. Ser. No. 10/407,846, which is hereby specifically incorporated by reference.

C. Oligonucleotide Hybridization

Oligonucleotides may be designed to hybridize directly to a target site of interest. The most common form of such analysis is where oligonucleotides are arrayed on a chip or plate in a “microarray.” Microarrays comprise a plurality of oligos spatially distributed over, and stably associated with, the surface of a substantially planar substrate, e.g., biochips. Microarrays of oligonucleotides have been developed and find use in a variety of applications, such as screening and DNA sequencing.

In gene analysis with microarrays, an array of “probe” oligonucleotides is contacted with a nucleic acid sample of interest, i.e., target. Contact is carried out under hybridization conditions and unbound nucleic acid is then removed. The resultant pattern of hybridized nucleic acid provides information regarding the genetic profile of the sample tested. Methodologies of gene analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. Various different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

D. Amplification of Nucleic Acids

In a particular embodiment, it may be desirable to amplify the target sequence before evaluating the SNP. Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA. The DNA also may be from a cloned source or synthesized in vitro.

The term “primer,” as used herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Pairs of primers designed to selectively hybridize to nucleic acids flanking the polymorphic site are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

It is also possible that multiple target sequences will be amplified in a single reaction. Primers designed to expand specific sequences located in different regions of the target genome, thereby identifying different polymorphisms, would be mixed together in a single reaction mixture. The resulting amplification mixture would contain multiple amplified regions, and could be used as the source template for polymorphism detection using the methods described in this application.

A number of template dependent processes are available to amplify the oligonucleotide sequences present in a given template sample. One of the best known amplification methods is the polymerase chain reaction (referred to as PCR™), which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed when the source of nucleic acid is fractionated or whole cell RNA. Methods of reverse transcribing RNA into cDNA are well known (see Sambrook et al., 1989). Alternative methods for reverse polymerization utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Another ligase-mediated reaction is disclosed by Guilfoyle et al. (1997). Genomic DNA is digested with a restriction enzyme and universal linkers are then ligated onto the restriction fragments. Primers to the universal linker sequence are then used in PCR to amplify the restriction fragments. By varying the conditions of the PCR, one can specifically amplify fragments of a certain size (i.e., less than a 1000 bases). An example for use with the present invention would be to digest genomic DNA with XbaI, and ligate on M13-universal primers with an XbaI over hang, followed by amplification of the genomic DNA with an M13 universal primer. Only a small percentage of the total DNA would be amplified (the restriction fragments that were less than 1000 bases). One would then use labeled primers that correspond to a SNP are located within XbaI restriction fragments of a certain size (<1000 bases) to perform the assay. The benefit to using this approach is that each individual region would not have to be amplified separately. There would be the potential to screen thousands of SNPs from the single PCR reaction, i.e., multiplex potential.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which are incorporated herein by reference in their entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include polymerization-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 discloses a nucleic acid amplification process involving cyclically synthesizing single-stranded RNA (ssRNA), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (ssDNA) followed by polymerization of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

Another advantageous step is to prevent unincorporated NTPs from being incorporated in a subsequent primer extension reaction. Commercially available kits may be used to remove unincorporated NTPs from the amplification products. The use of shrimp alkaline phosphatase to destroy unincorporated NTPs is also a well-known strategy for this purpose.

E. Sequencing

DNA sequencing enables one to perform a thorough analysis of DNA because it provides the most basic information of all: the sequence of nucleotides. Maxam & Gilbert developed the first widely used sequencing methods—a “chemical cleavage protocol.” Shortly thereafter, Sanger designed a procedure similar to the natural process of DNA replication. Even though both teams shared the 1980 Nobel Prize, Sanger's method became the standard because of its practicality.

Sanger's method, which is also referred to as dideoxy sequencing or chain termination, is based on the use of dideoxynucleotides (ddNTP's) in addition to the normal nucleotides (NTP's) found in DNA. Dideoxynucleotides are essentially the same as nucleotides except that they contain a hydrogen group on the 3′ carbon instead of a hydroxyl group (OH). These modified nucleotides, when integrated into a sequence, prevent the addition of further nucleotides. This occurs because a phosphodiester bond cannot form between the dideoxynucleotide and the next incoming nucleotide, and thus the DNA chain is terminated. Using this method, optionally coupled with amplification of the nucleic acid target, one can now rapidly sequence large numbers of target molecules, usually employing automated sequencing apparati. Such techniques are well known to those of skill in the art.

F. Other Techniques

There are a variety of ways by which one can assess genetic profiles, and may of these rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

Various nucleic acids may be visualized in order to confirm their presence, quantity or sequence. In one embodiment, the primer is conjugated to a chromophore but may instead be radiolabeled or fluorometrically labeled. In another embodiment, the primer is conjugated to a binding partner that carries a detectable moiety, such as an antibody or biotin. In other embodiments, the primer incorporates a fluorescent dye or label. In yet other embodiments, the primer has a mass label that can be used to detect the molecule amplified. Other embodiments also contemplate the use of Taqman™ and Molecular Beacon™ probes. Alternatively, one or more of the dNTPs may be labeled with a radioisotope, a fluorophore, a chromophore, a dye or an enzyme. Also, chemicals whose properties change in the presence of DNA can be used for detection purposes. For example, the methods may involve staining of a gel with, or incorporation into the separation media, a fluorescent dye, such as ethidium bromide or Vistra Green, and visualization under an appropriate light source.

The choice of label incorporated into the products is dictated by the method used for analysis. When using capillary electrophoresis, microfluidic electrophoresis, HPLC, or LC separations, either incorporated or intercalated fluorescent dyes are used to label and detect the amplification products. Samples are detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin:avidin interaction, following separation of the amplification products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer no label is required because nucleic acids are detected directly.

In the case of radioactive isotopes, tritium, ¹⁴C and ³²P are used predominantly. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference in its entirety.

The present invention relies on the use of agents that are capable of detecting single nucleotide changes in DNA. These agents generally fall into two classes—agents that hybridize to target sequences that contain the change, and agents that hybridize to target sequences that are adjacent to (e.g., upstream or 5′ to) the region of change. A third class of agents, restriction enzymes, do not hybridize, but instead cleave at a target site. A list of restriction enzymes can be found on the world-wide-web at fermentas.com/techinfo/re/prototypes.htm, hereby incorporated by reference.

Oligonucleotide synthesis is well known to those of skill in the art. Various mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference in its entirety. Basically, chemical synthesis can be achieved by the diester method, the triester method polynucleotides phosphorylase method and by solid-phase chemistry. These methods are discussed in further detail below.

Diester Method.

The diester method was the first to be developed to a usable state, primarily by Khorana and co-workers (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester Method.

The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore, purifications are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide Phosphorylase Method.

This is an enzymatic method of DNA synthesis that can be used to synthesize many useful oligodeoxynucleotides (Gillam et al., 1978). Under controlled conditions, polynucleotide phosphorylase adds predominantly a single nucleotide to a short oligodeoxynucleotide. Chromatographic purification allows the desired single adduct to be obtained. At least a trimer is required to initiate the method of adding one base at a time, a primer that must be obtained by some other method. The polynucleotide phosphorylase method works and has the advantage that the procedures involved are familiar to most biochemists.

Solid-Phase Methods.

The technology developed for the solid-phase synthesis of polypeptides has been applied after an, it has been possible to attach the initial nucleotide to solid support material has been attached by proceeding with the stepwise addition of nucleotides. All mixing and washing steps are simplified, and the procedure becomes amenable to automation. These syntheses are now routinely carried out using automatic DNA synthesizers.

Phosphoramidite chemistry (Beaucage, 1993) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

In certain embodiments, nucleic acid products are separated by agarose, agarose-acrylamide or polyacrylamide gel electrophoresis using standard methods (Sambrook et al., 1989). Separated products may be cut out and eluted from the gel for further manipulation. Using low melting point agarose gels, the skilled artisan my remove the separated band by heating the gel, followed by extraction of the nucleic acid.

Separation of nucleic acids may also be effected by chromatographic techniques known in the art. There are many kinds of chromatography that may be used in the practice of the present invention, including capillary adsorption, partition, ion-exchange, hydroxylapatite, molecular sieve, reverse-phase, column, paper, thin-layer, and gas chromatography as well as HPLC.

A number of the above separation platforms can be coupled to achieve separations based on two different properties. For example, some of the primers can be coupled with a moiety that allows affinity capture, and some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

### IV. SYSTEMIC LUPUS ERYTHEMATOSUS

### A. Definition and Symptoms

Systemic Lupus Erythematosus (SLE) is an autoimmune chronic inflammatory disease that most commonly affects the skin, joints, kidneys, heart, lungs, blood vessels, and brain. The most common symptoms include fatigue, muscle aches, low-grade fever, skin rashes, and kidney problems that are sometimes severe enough to require dialysis or transplant. Symptoms may also include a characteristic facial rash (“butterfly rash”), photosensitivity, and poor circulation to the extremities with cold exposure, known as Raynaud's phenomenon. Rheumatoid arthritis is another chronic autoimmune disease, and most people with SLE will develop arthritis during the course of their illness with similar symptoms to rheumatoid arthritis. Because SLE can affect the walls of the blood vessels, young women with SLE are at significantly higher risk for heart attacks from coronary artery disease. For many patients, alopecia occurs as SLE worsens.

Women who become pregnant with SLE are considered “high risk.” These women have an increased risk of miscarriages, and the incidence of flares can increase with pregnancy. Antibodies from SLE can be transferred to the fetus, resulting in “neonatal lupus.” Symptoms of neonatal lupus include anemia and skin rash, with congenital heart block being less common. Unlike SLE, neonatal lupus resolves after six months as the newborn metabolizes the mother's antibodies.

### B. Diagnosis

Because the symptoms of SLE can vary widely, accurate diagnosis is difficult. A diagnosis of SLE is suggested for a patient who meets four or more of the eleven criteria established by the American Rheumatism Association, but there is currently no single test that establishes the diagnosis of SLE. However, these criteria are not definitive. The criteria are based on the symptoms of SLE, but also include the presence of anti-DNA, antinuclear (ANA), or anti-Sm antibodies, a false positive test for syophilis, anticardiolipin antibodies, lupus anticoagulant, or positive LE prep test. Some patients are diagnosed with SLE who manifest fewer than four criteria, while other such patients remain undiagnosed.

Most people with SLE test positive for ANA. Even so, the test is not definitive, as a number of conditions can cause a positive ANA test. Other antibody tests that can aid in a diagnosis of SLE or other autoimmune conditions include anti-RNP, anti-Ro (SSA), and anti-La (SSB).

### C. Treatment

There is currently no cure for SLE, and the illness remains characterized by alternating periods of illness, or flares, and periods of wellness, or remission. The current goal of treatment is to relieve the symptoms of SLE, and to protect the organ systems affected by decreasing the level of autoimmune activity. More and better quality rest is prescribed for fatigue, along with exercise to maintain joint strength and range of motion. DHEA (dehydroepiandrosterone) can reduce fatigue and thinking problems associated with SLE. Physicians also commonly prescribe Nonsteroidal antiinflammatory drugs (NSAIDs) for pain and inflammation, although this can cause stomach pain and even ulcers in some patients.

Hydroxychloroquine, an anti-malarial medication, can be effective in treating fatigue related to SLE as well as skin and joint problems. Hydroxychloroquine also decreases the frequency of excessive blood clotting in some SLE patients. Corticosteroids are needed for more serious cases, although the serious side effects, such as weight gain, loss of bone mass, infection, and diabetes limits the length of time and dosages at which they can be prescribed. Immunosuppressants, or cytotoxic drugs, are used to treat severe cases of SLE, but again serious side effects such as increased risk of infection from decreased blood cell counts are common.

Possible future therapies include stem cell transplants to replace damaged immune cells and radical treatments that would temporarily kill all immune system cells. Other future treatments may include “biologic agents” such as the genetically engineered antibody rituximab (anti-CD20) that block parts of the immune system, such as B cells. Recently, two groups of researchers found that even partial restoration of function of an inhibitory Fc receptor prevented the development of SLE in several strains of mice that were genetically prone to the disease. Reviewed in Kuehn, Lupus (2005).

### D. Who SLE Affects

SLE is much more common among women than men, with women comprising approximately 90% of all SLE patients. It is also three times more common in African American women than in women of European descent, although the incidence is also higher among women of Japanese and Chinese ancestry.

Because widely varying symptoms of SLE make accurate diagnosis difficult, the exact number of people who suffer from SLE is unknown. The Lupus Foundation of America, however, estimates that approximately 1,500,000 Americans have some form of lupus. The prevalence of SLE is estimated to be about 40 per 100,000.

### V. KITS

All the essential materials and reagents required for detecting SNPs in a sample may be assembled together in a kit. This generally will comprise a primer or probe designed to hybridize specifically to or upstream of target nucleotides of the polymorphism of interest. The primer or probe may be labeled with a radioisotope, a fluorophore, a chromophore, a dye, an enzyme, or TOF carrier. Also included may be enzymes suitable for amplifying nucleic acids, including various polymerases (reverse transcriptase, Taq, etc.), dNTPs/rNTPs and buffers (e.g., 10× buffer=100 mM Tris-HCl (pH 8.3), and 500 mM KCl) to provide the necessary reaction mixture for amplification. One or more of the deoxynucleotides may be labeled with a radioisotope, a fluorophore, a chromophore, a dye, or an enzyme. Such kits may also include enzymes and other reagents suitable for detection of specific nucleic acids or amplification products.

The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods

Genome-Wide Association Scan.

The genotyping, quality control, data analysis procedures, and summary statistics for the GWA study were described previously in Graham et al. (2008).¹⁹

Study Design.

The genotype data used in this study were generated as a part of a joint effort of more than 40 investigators from around the world who contributed samples, funding and hypotheses on a combined array containing ˜35,000 SNPs (data not shown). The Oklahoma Medical Research Foundation (OMRF) served as the coordinating center, ran the arrays, and sent the data to a central quality control center at Wake Forest Medical Center. These data were then distributed back to the investigators who requested the SNPs for final analysis and publication.²³⁻²⁸

Subjects.

The multi-racial replication study consisted of 17,003 total samples (8,922 SLE cases and 8,077 controls) and included individuals of self-reported African-American, Asian, European, Gullah, Hispanic, and Amerindian ancestry (data not shown). A total of 374 samples were common between the GWA scan and the replication study to confirm genotypes generated by the two platforms and to obtain genotypes at SNPs not present on the Affymetrix 5.0 array. These data were only used as observed data for the imputation analysis of specific genomic regions, as described below; to maintain independence between the GWA and replication samples, the data generated on these shared samples were not included in the replication or fine-mapping analyses. The OMRF gathered the samples from properly consented subjects (following the guidelines of the ethics committees at the respective institutions where they were collected) and prepared them for genotyping. All cases used in this study fulfilled at least 4 of the 11 American College of Rheumatology criteria for SLE, while healthy, population-based controls were without family history of SLE or any other autoimmune disease.²⁹

Genotyping and Sample Quality Control.

A total of 1580 SNPs that attained p<0.05 in the previously published GWA scan were selected for replication. In addition, 287 SNPs within the IRF8 region (chosen to capture all variation with a minimum r² threshold of 0.8 using the TAGGER algorithm in HAPLOVIEW³⁰) and 347 ancestral-informative markers (AIMs) spanning the genome were genotyped. SNPs were genotyped at OMRF using Infinium chemistry on an Illumina iSelect custom array following the manufacturer's protocol. The following quality control procedures were implemented prior to analysis (data not shown): well-defined clusters within the scatter plots, SNP call rate >90% across all samples genotyped, minor allele frequency >1%, sample call rate >90%, p>0.05 for differential missingness between cases and controls, total proportion missing <5%, and Hardy-Weinberg proportions (HWP) with p>0.01 in controls and p>0.0001 in cases.

Samples exhibiting excess heterozygosity (>5 standard deviations from the mean) or <90% call rate were excluded from the analysis. The remaining individuals were examined for excessive allele sharing as estimated by identity-by-descent (IBD). In sample pairs with excess relatedness (IBD >0.4), one individual was removed from the analysis using the following criteria: 1) remove sample with lower call rate, 2) remove control and retain case, 3) remove male sample before female, 4) remove younger control before older, and 5) in the situation with two cases, remove case with less phenotype data available. Discrepancies between self-reported and genetically determined gender were evaluated. Males were required to be heterozygous at rs2557523 (since the G allele for this SNP is only observed on the Y chromosome and the A allele appears only on the X chromosome) and to have chromosome X heterozygosity ≦10%. Females were required to be homozygous for the A allele at rs2557523 and to have chromosome X heterozygosity >10%.

Ascertainment of Population Stratification.

Genetic outliers from each ethnic and/or racial group were removed from further analysis as determined by principal components analysis and admixture estimates (data not shown).^{31,32} Population substructure was identified within the sample set using both EIGENSTRAT³¹ and ADMIXMAP^{33,34} using the 163 AIMs that passed quality control to distinguish the four continental ancestral populations: Africans, Europeans, Amerindians, and East Asians (data not shown).^{35,36} The inventors utilized principal components from EIGENSTRAT outputs to identify outliers >4 standard deviations from the mean of each of the first three principal components (PC) for the individual population clusters. After quality control, a total of 1,139 samples were excluded (data not shown). Overall, 2,586 subjects were included in the GWA scan and 15,490 subjects were included in the replication study, giving a total of 18,076 subjects.

Statistical Analysis.

To test for SNP-SLE association in the replication study, logistic regression was computed as implemented in PLINK ver. 1.07.³⁷ The additive genetic model was calculated while adjusting for the first three PCs and gender. Models were also adjusted for ancestry using estimates provided by ADMIXMAP and resulted in no observable difference in association as compared with PC adjustment. Conditional likelihood ratio tests were conducted using the extended WHAP functionality in PLINK ver. 1.07. The genome-wide p-value threshold for all data replicating GWA results was p<5×10⁻⁸ after meta-analysis. For the fine-mapping and imputation of IRF8, the inventors utilized a Bonferroni corrected p-value threshold of p<1.09×10⁻⁴ based on the maximum number of tests across all populations (460 independent variants with r²<0.8). Meta-analyses of the SNPs observed in both the GWA scan and the multi-racial replication study were calculated with a weighted Z-score using METAL.³⁸ Each racial group was weighted by the square root of its sample size to control for sample size differences between studies. Unless noted, the inventors combined all data generated unless the variant failed quality control in a given racial group.

To test for meta-analysis heterogeneity, the inventors utilized both the Cochran's Q test statistic and I² index. The Cochran's Q is a classical method that calculates the weighted sum of the squared deviations between individual study effects and the overall effect across studies.³⁹ It follows a chi-square distribution with k−1 degrees of freedom, where k is the number of studies. A value of p<0.05 was considered significant evidence for heterogeneity. The I² index measures the degree or percentage of inconsistency across studies due to heterogeneity rather than random chance.⁴⁰ The I² index ranges between 0% and 100%, where I² equal 0% to 25%, 26% to 50%, 51% to 75%, and 76% to 100%, indicating low, moderate, high, and very high heterogeneity, respectively.

Linkage disequilibrium and probable haplotypes were determined using HAPLOVIEW ver. 4.2.³⁰ Haplotype blocks were calculated for those haplotypes present at >3% frequency using the solid-spine of LD algorithms with minimum r² values of 0.8.³⁰

Resequencing.

The inventors resequenced the IRF8 region (Chr 16, 84,488,150-84,539,352 bp) in 206 (92 SLE cases and 114 healthy controls) European and 46 (25 SLE cases and 21 healthy controls) African-American subjects. For each sample, 3-5 μg of whole genomic DNA were sheared and prepared for sequencing using an Illumina Paired-End Genomic DNA Sample Prep Kit. Targeted regions of interest from each sample were then enriched with a SureSelect Target Enrichment System utilizing a custom-designed bait pool (Agilent Technologies). Resequencing was undertaken using an Illumina GAIIx platform employing standard procedures. Post-sequence data were processed with Illumina's Pipeline software v.1.7. All samples were sequenced to minimum average fold coverage of 25×.

Variant Detection and Quality Control.

Unique sequences corresponding to an individual nucleotide molecule were aligned to the human genome reference (hg18) using the Burrows-Wheeler Aligner (BWA) alignment tool.⁴¹ Following initial alignment, reads were locally re-aligned around known and suspected insertion and deletion sites using the Genome Analysis Toolkit (GATK) analysis suite to generate the best possible read alignment.⁴² The GATK suite was then used empirically to re-calculate the correct quality score for each base within the alignment. This process served to correct overestimated high-quality scores initially reported by the sequencer itself.

Following local re-alignment around deletion-insertion polymorphism (DIP) sites and base quality score recalibration, SNP and DIP genotypes were generated for each sample individually as well as for the samples as a whole. Finally, SNP and DIP genotypes were hard-filtered against a set of criteria designed to remove any remaining low quality calls. For a variant to be included in the call list, the inventors required a Phred quality score >30, a quality by depth ratio of >5.0, a strand bias score of <−0.10, and a homopolymer run of <5 bases. The program BEAGLE was used to determine the variant phase.⁴³ Variants meeting call parameters were output to files compatible with PLINK and other genotyping tools utilizing the VCFtools analysis suite.

To assess the accuracy of sequence-based SNP calling, the inventors cross-referenced the sequenced and genotyped allele calls. The inventors observed ˜99% concordance between genotypes and sequence-based variant detection, suggesting high-quality sequence data. Samples with ≧5% of variants differing between sequencing and genotyping were manually inspected to determine where sequence quality was poor. As an additional quality control measure, each variant identified by the automated workflow was confirmed by manual inspection of the assembled contig using the Integrative Genomics Viewer (IGV) program.⁴⁴

Imputation.

To increase the informativeness of the IRF8 region, imputation was conducted in subjects of European, African-American, and Asian ancestry over a 100-kB interval spanning the IRF8 locus. Imputation of the replication data across chromosome 16 (84.45 Mb-84.46 MB) was performed using IMPUTE2 and the reference panels provided in Supplemental Table 7.⁴⁵⁻⁴⁷ Imputed genotypes were required to meet or exceed a probability threshold of 0.8, an information measure of >0.4, and the same quality control criteria thresholds described above for inclusion in the analyses.

### Example 2

### Results

Two SNPs, rs11648084 and rs11644034, telomeric to interferon regulatory factor 8 (IRF8) at 16q24.1 were suggestive for association in the inventors' published GWA study (p=5.99×10⁻⁴ and 2.29×10⁻³, respectively; OR=0.76 and 0.66, respectively; Table 1). Both rs11648084 and rs11644034 were replicated in the current, independent population of SLE cases and controls of European ancestry and exceeded the genome-wide threshold (p_{meta-Euro}=2.34×10⁻⁹ and p_{meta-Euro}=2.08×10⁻¹⁰, respectively; Table 1). However, neither SNP was significantly associated with SLE in any other population studied likely due to the reduced sample size, clinical and/or genetic heterogeneity, decreased minor allele frequency, and/or reduced correlation with the causal variants, which is also reflected by the test of heterogeneity for these SNPs (Table 1 and data not shown).

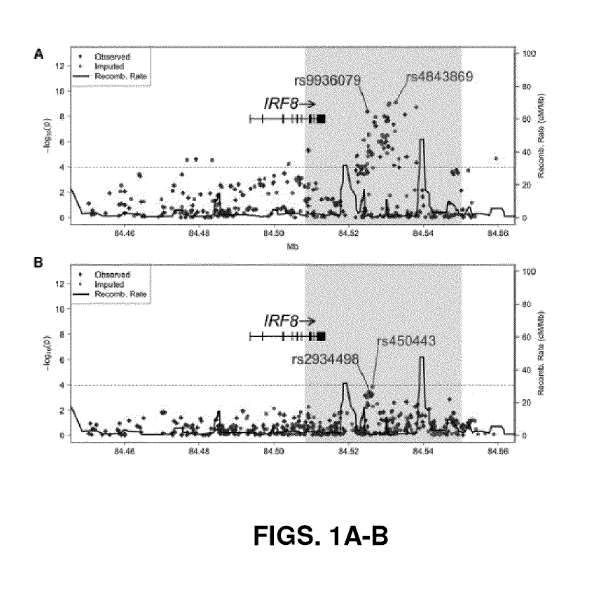

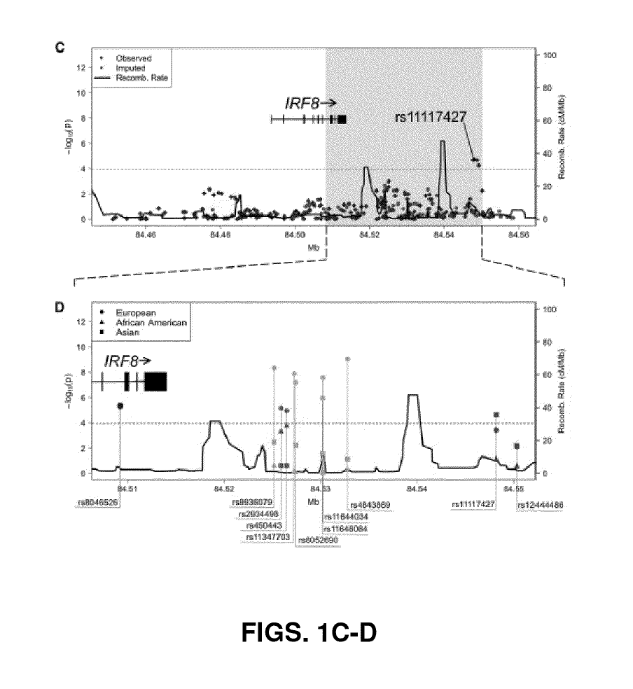

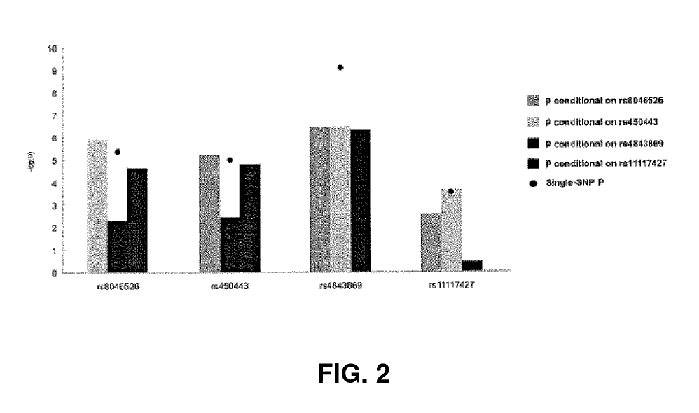

To better refine the association signal, 287 additional SNPs covering ˜100 kB encompassing the IRF8 coding region were genotyped (see Methods; FIGS. 1A and 1D, Table 2, and data not shown). The most significant association in the European population was with rs9936079 (p=3.96×10⁻⁹, OR=0.77; FIGS. 1A and 1D and Table 2) located ˜11 kB telomeric to IRF8 and found to be in strong linkage disequilibrium (LD) with rs11644034 (r²=0.92; FIGS. 2B-C). The Asian population also exhibited association with rs9936079 (p=2.95×10⁻³, OR=0.73); however, rs9936079 failed to pass quality control measures (see Methods; differential missingness p=10⁻⁷) in individuals of African ancestry and was not associated with disease in patients of Hispanic or Amerindian ancestry. Meta-analysis yielded pₘₑₜₐ₋ₐₗₗ=9.28×10⁻¹¹ (Table 2 and data not shown).

The inventors observed a modest association in the African-Americans at rs2934498 (p=3.92×10⁻⁴, OR=0.83), which was also significant in those individuals of European ancestry (p=5.96×10⁻⁶, OR=1.19), but not in those of Asian ancestry (FIGS. 1B and 1D, Table 2, and data not shown). The strongest Asian association was observed in a region ˜34 kB telomeric to IRF8 (rs11117427, p=1.99×10⁻⁵, OR=0.64; FIGS. 1C and 1D, Table 2, and data not shown). Association was also observed with rs11117427 in the Europeans (p=3.46×10⁻⁴, OR=0.84; FIGS. 1A and 1D and Table 2). Interestingly, this SNP is only ˜2 kB from rs12444486, which has been reported by Gateva et al. as being suggestive for association with SLE in Europeans (FIG. 1D and data not shown).²²

Resequencing of the IRF8 region was performed in 206 subjects of European and 46 subjects of African-American ancestry to identify variants not previously evaluated within the IRF8 region and to assess their association to SLE (see Methods). Thirty-eight and 85 variants not present in dbSNP 130 were identified in European and African-American individuals, respectively. After imputing these data into the larger European and African-American datasets (see Methods), the most significantly associated region within the European population was ˜19 kB telomeric to IRF8 (rs4843869, p=7.61×10⁻¹⁰, OR=0.76; Table 2 and FIGS. 1A and 1D). Ultimately, three strongly correlated (r²>0.90) SNPs emerged as the most significantly associated with SLE in the Europeans: rs11644034 (identified via GWA), rs9936079 (identified by fine-mapping), and rs4843869 (imputed based on resequencing). Interestingly, targeted resequencing revealed a DIP (rs11347703, p=1.11×10⁻⁸, OR=0.78) that was located less than 100 bp from a genotyped SNP, rs8052690 (p=5.69×10⁻⁸, OR=0.79, Table 2). This DIP, with high biological plausibility, was in strong LD with the peak European SNP (rs4843869) and rs8052690 (r²/D′>0.9; FIG. 2). Of note, some of the African-Americans resequenced in this study did harbor the DIP, rs11347703, identified in the Europeans. However, neither rs11347703 nor any SNP correlated with it was found to be significantly associated with SLE in African-Americans, likely due to the decrease in power and/or a decrease in the minor allele frequency.

The peak association in African-Americans following imputation was at rs450443 (p=1.41×10⁻⁴, OR=0.82; Table 2 and FIGS. 1B and 1D) and was in strong LD (r²=0.88 with rs2934498. Patients of European, but not Asian, ancestry showed association with rs450443 (p=9.73×10⁻⁶, OR=1.18; Table 2 and FIGS. 1A, 1C and 1D). Imputation was conducted in the Asian population using 1000 Genomes phased haplotypes⁴⁸; however, rs11117427 remained the peak signal (Table 2 and FIGS. 1C and 1D).

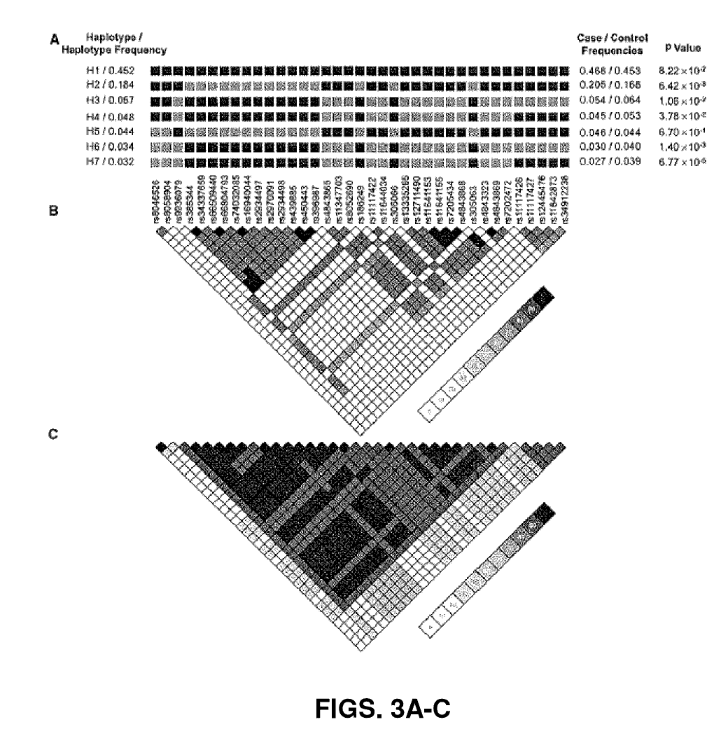

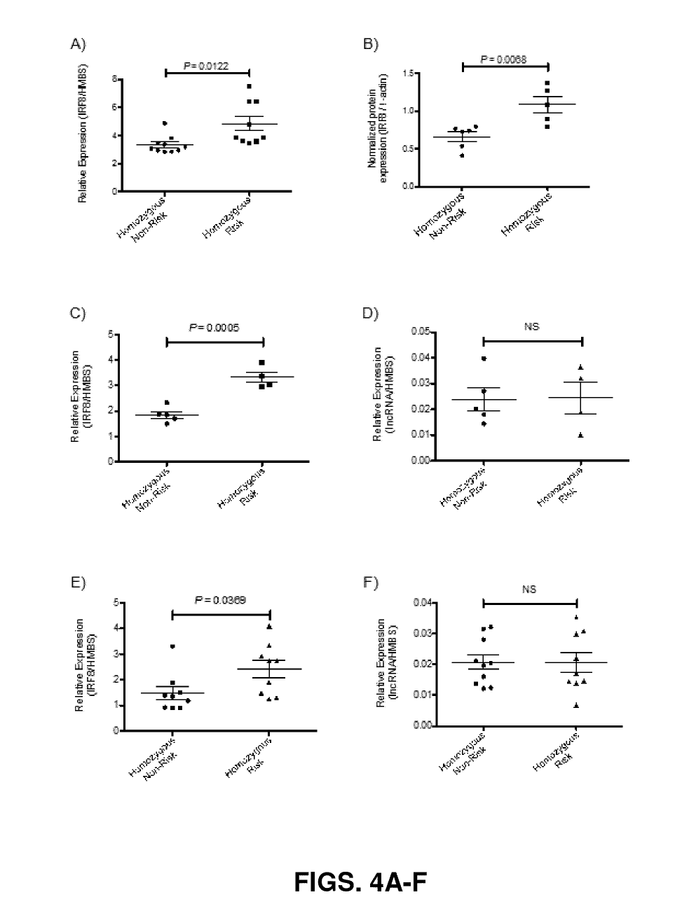

To assess the independence of variants in the European population, the inventors used logistic regression models adjusting for the best tagging SNPs at each signal. When the inventors adjusted for rs4843869 in Europeans, the association persisted at rs450443 and variants correlated to it. However, adjusting for rs4843869 negated the association with rs11117427 and its correlated variants (FIGS. 3A-C, FIG. 2, and data not shown). Adjusting for either rs450443 or rs11117427 was only able to negate the associations of the polymorphisms that were correlated with each of these SNPs (FIGS. 3A-C, FIG. 2, and data not shown). A SNP in the 6^{th} intron of IRF8, rs8046526, was also associated with SLE risk in Europeans (p=3.96×10⁻⁶, OR=0.80) and remained significant after adjusting for the other SNPs (Table 2, FIGS. 1A and 1D, FIGS. 3A-C, FIG. 2, and data not shown). Adjusting for rs8046526 in the Europeans only negated associations for itself and its correlated variants. However, adjusting the logistic regression model for rs8046526, rs450443, and rs4843869 negated all associations present in the European population (FIG. 3, FIGS. 2B-C, and data not shown), demonstrating the importance of these three IRF8 variants for SLE risk.

Haplotype analysis identified a single risk haplotype (H2) (p=6.42×10⁻⁸) with a frequency of 18.4% in the European individuals (FIG. 2; FIGS. 3A-C). Two significant protective haplotypes, H6 and H7, were also identified (FIG. 2; FIGS. 3A-C). The risk-associated alleles within the region bounded by SNPs rs11117426 to rs34912238 (the peak Asian effect) were also present in the most significant protective haplotype, H7, suggesting that this region likely does not impact disease risk in Europeans (FIG. 2; FIGS. 3A-C). The only difference between H3/H6 and H4/H7 are rs8046526 and rs8058904 in the minor form suggesting that these SNPs are important in conferring protection from disease (FIG. 2; FIGS. 3A-C). The only differences between H2 and H5 (which are not statistically significant) are the major alleles for SNPs rs8046526 and rs8058904 residing on the H2 haplotype and the minor alleles on the neutral H5 haplotype. Thus, it appears that all three regions (tagged by rs8046526, rs450443, and rs4843869) are required for risk. Many variants residing on the risk haplotype are within regions known to bind multiple transcription factors in the ENCODE ChIP-Seq project dataset in immunologic cell types (data not shown).⁴⁹ Thus, the inventors hypothesize that the risk haplotype likely affects the regulation of IRF8 expression and/or other genes in the region.

A coding SNP (rs1132200) within transmembrane protein 39A (TMEM39A) at 3q13.33 that demonstrated suggestive evidence of association in the inventors' previous GWA scan (p=1.65×10⁻³), was also confirmed in the European replication study (p=2.37×10⁻⁴, OR=0.83; Table 1, and data not shown). This non-synonymous SNP showed association with SLE in Asian patients (p=1.66×10⁻³, OR=0.73), but not in African-Americans, Hispanics, Gullah, or Amerindians (Table 1 and data not shown). When analyzing this SNP in all populations that passed quality control, a meta-analysis produced pₘₑₜₐ₋ₐₗₗ=8.62×10⁻⁹, and no evidence of heterogeneity was observed between these datasets (Table 1, and data not shown).

Finally, the inventors replicated several SNPs in the region of the IKAROS family of zinc finger 3 AIOLOS (IKZF3) gene and the zona pellucida binding protein 2 (ZPBP2) gene on chromosome 17q12 (Table 1, and data not shown). Three SNPs within IKZF3 replicated, with rs8079075 being the most significant SNP in both the samples of European (p=5.08×10⁻⁴, OR=1.39) and African-American (p=2.62×10⁻³, OR=1.26) ancestry (pₘₑₜₐ₋ₐₗₗ=4.83×10⁻⁹). The most significant SNP in this region (rs1453560) is located between IKZF3 and ZPBP2 and was replicated in European (p=6.42×10⁻⁴, OR=1.37) and African-American (p=4.86×10⁻⁴, OR=1.23) ancestral populations, resulting in pₘₑₜₐ₋ₐₗₗ=3.48×10⁻¹⁰ (Table 1, and data not shown). All four SNPs are highly correlated (r²>0.95). Even though the Cochran's Q test of heterogeneity was not statistically significant, the inventors observed moderate heterogeneity by the I² index likely due to the differences in allele frequency between the racial groups (Table 1). IKZF3 and ZPBP2 are transcribed in opposite directions of one another, but share the same promoter region (data not shown). The ENCODE ChIP-Seq project has identified multiple transcription factor binding sites for chromatin in the chromosomal region surrounding rs1453560 (data not shown).⁴⁹

In addition to the three regions described above that now exceed genome-wide significance, 11 loci were replicated in the European SLE cases, but did not exceed genome-wide significance (5×10⁻⁸<p_{meta-Euro}<9.99×10⁻⁵), including: CFHR1 (MIM 134371), CADM2, LOC730109/IL12A (MIM 161560), LPP (MIM 600700), LOC63920, SLU7 (MIM 605974), ADAMTSL1 (MIM 609198), C10orf64, OR8D4, FAM19A2, and STXBP6 (MIM 607958) (Table 3 and data not shown).

TABLE 1
SLE Risk Loci Surpassing the Genome-wide Significance Threshold^{a}

| | | | | European (3,562 Cases/3,491 Controls) | | | | |
|---|---|---|---|---|---|---|---|---|
| Chr | SNP | Locus | Alleles^{b} | p_{GWA scan}^{c} | OR_{GWA scan} (95% CI) | p_{REP} | OR_{REP} (95% CI) | p_{META-Euro} |
| 3 | rs1132200 | TMEM39A | G/A | 1.65 × 10⁻³ | 0.72 | 2.37 × 10⁻⁴ | 0.83 | 1.81 × 10⁻⁵ |
| | | | | | (0.59-0.88) | | (0.76-0.92) | |
| 16 | rs11644034 | IRF8 | G/A | 2.29 × 10⁻³ | 0.66 | 2.36 × 10⁻⁸ | 0.78 | 2.08 × 10⁻¹⁰ |
| | | | | | (0.54-0.79) | | (0.71-0.85) | |
| 16 | rs11648084 | IRF8 | G/A | 5.99 × 10⁻⁴ | 0.76 | 9.35 × 10⁻⁷ | 0.83 | 2.34 × 10⁻⁹ |
| | | | | | (0.65-0.89) | | (0.77-0.89) | |
| 17 | rs9913957 | IKZF3 | A/G | 7.87 × 10⁻³ | 1.75 | 5.14 × 10⁻⁴ | 1.38 | 1.38 × 10⁻⁵ |
| | | | | | (1.27-2.41) | | (1.15-1.66) | |
| 17 | rs8076347 | IKZF3 | C/A | 3.07 × 10⁻³ | 1.93 | 3.04 × 10⁻³ | 1.32 | 4.75 × 10⁻⁶ |
| | | | | | (1.41-2.62) | | (1.10-1.58) | |
| 17 | rs8079075 | IKZF3 | A/G | 1.47 × 10⁻³ | 1.90 | 5.08 × 10⁻⁴ | 1.39 | 3.81 × 10⁻⁶ |
| | | | | | (1.39-2.59) | | (1.16-1.68) | |
| 17 | rs1453560 | ZPBP2 | A/C | 7.81 × 10⁻⁴ | 1.92 | 6.42 × 10⁻⁴ | 1.37 | 3.21 × 10⁻⁶ |
| | | | | | (1.41-2.61) | | (1.14-1.64) | |

| | African American | | | | | | |
|---|---|---|---|---|---|---|---|
| | (1,527 Cases/1,811 Controls) | | Asian (1,265 Cases/1,260 Controls) | | Meta | Test of Heterogeneity | |
| Chr | p | OR (95% CI) | p | OR (95% CI) | p_{META-ALL}^{d} | p [symbol] | t [symbol] |
| 3 | 6.92 × 10⁻² | 0.75 | 1.66 × 10⁻³ | 0.73 | 8.62 × 10⁻⁹ | 0.450 | 0.0% |
| | | (0.56-1.02) | | (0.59-0.89) | | | |
| 16 | 5.10 × 10⁻¹ | 0.95 | 2.63 × 10⁻² | 0.79 | 2.72 × 10⁻⁹ | 0.016 | 61.5% |
| | | (0.81-1.11) | | (0.65-0.97) | | | |
| 16 | 6.33 × 10⁻² | 0.90 | 7.52 × 10⁻¹ | 1.02 | 7.00 × 10⁻⁷ | 0.001 | 74.0% |
| | | (0.81-1.01) | | (0.91-1.14) | | | |
| 17 | 1.07 × 10⁻² | 1.22 | — | — | 1.39 × 10⁻⁸ | 0.105 | 45.1% |
| | | (1.05-1.41) | | | | | |
| 17 | 2.19 × 10⁻³ | 1.20 | — | — | 3.01 × 10⁻⁸ | 0.047 | 55.4% |
| | | (1.07-1.34) | | | | | |
| 17 | 2.62 × 10⁻³ | 1.26 | — | — | 4.83 × 10⁻⁹ | 0.201 | 31.3% |
| | | (1.08-1.46) | | | | | |
| 17 | 4.86 × 10⁻⁴ | 1.23 | — | — | 3.48 × 10⁻¹⁰ | 0.097 | 46.4% |
| | | (1.09-1.37) | | | | | |

The following abbreviations are used: Chr, chromosome; OR, odds ratio; GWA, genome-wide association; REP, replication; and CI, confidence interval.
^{a}Table S7 contains results for all populations evaluated within this study.
^{b}Major/minor alleles.
^{c}Result of GWA scan was previously reported in Graham et. al. [symbol]
^{d}Data were combined for all racial groups genotyped within our study that passed quality control.
^{e}Cochran's Q test statistic. [symbol]
indicates data missing or illegible when filed

TABLE 2
IRF8 Variants Associated with SLE^{a}

| | Genotyped | | European | | | | | |
|---|---|---|---|---|---|---|---|---|
| SNP | or Imputed | Position (bp) | Alleles^{b} | MAF^{c} | p | OR (95% CI) | p_{African-American} | p_{Asian} |
| rs8046526 | I | 84,509,136 | C/T | 0.14/0.16 | 3.96 × 10⁻⁶ | 0.80 (0.73-0.88) | — | — |
| rs8058904 | G | 84,509,183 | A/G | 0.14/0.16 | 5.14 × 10⁻⁶ | 0.80 (0.73-0.88) | 1.96 × 10⁻¹ | — |
| rs9936079 | G | 84,525,095 | G/A | 0.17/0.22 | 3.96 × 10⁻⁹ | 0.77 (0.70-0.84) | — | 2.95 × 10⁻³ |
| rs385344 | I | 84,525,105 | C/G | 0.30/0.27 | 1.37 × 10⁻⁵ | 1.18 (1.10-1.27) | — | 2.43 × 10⁻¹ |
| rs34337659 | I | 84,525,158 | T/C | 0.31/0.27 | 1.55 × 10⁻⁵ | 1.18 (1.10-1.27) | 3.97 × 10⁻⁴ | 1.14 × 10⁻¹ |
| rs66509440 | I | 84,525,182 | C/T | 0.28/0.25 | 6.36 × 10⁻⁶ | 1.20 (1.11-1.30) | 3.97 × 10⁻⁴ | — |
| rs66804793 | I | 84,525,190 | G/A | 0.28/0.25 | 6.16 × 10⁻⁶ | 1.20 (1.11-1.30) | 3.84 × 10⁻⁴ | — |
| rs74032085 | I | 84,525,245 | T/C | 0.28/0.24 | 2.25 × 10⁻⁶ | 1.22 (1.12-1.32) | 4.72 × 10⁻⁴ | — |
| rs16940044 | I | 84,525,266 | A/G | 0.27/0.24 | 2.16 × 10⁻⁶ | 1.22 (1.12-1.32) | 4.73 × 10⁻⁴ | — |
| rs2934497 | I | 84,525,379 | C/T | 0.27/0.23 | 2.62 × 10⁻⁷ | 1.24 (1.14-1.35) | 5.12 × 10⁻⁴ | 1.95 × 10⁻¹ |
| rs2970091 | I | 84,525,387 | G/A | 0.28/0.24 | 2.96 × 10⁻⁷ | 1.24 (1.14-1.34) | 5.31 × 10⁻⁴ | 1.93 × 10⁻¹ |
| rs2934498 | G | 84,525,783 | A/G | 0.31/0.27 | 5.96 × 10⁻⁶ | 1.19 (1.11-1.29) | 3.92 × 10⁻⁴ | 2.09 × 10⁻¹ |
| rs439885 | G | 84,526,175 | G/A | 0.31/0.27 | 1.16 × 10⁻⁵ | 1.19 (1.10-1.28) | 5.59 × 10⁻⁴ | 1.98 × 10⁻¹ |
| rs450443 | I | 84,526,392 | T/G | 0.30/0.27 | 9.73 × 10⁻⁶ | 1.18 (1.10-1.28) | 1.41 × 10⁻⁴ | 2.04 × 10⁻¹ |
| rs396987 | I | 84,526,435 | A/G | 0.30/0.27 | 8.89 × 10⁻⁶ | 1.19 (1.10-1.28) | 5.44 × 10⁻⁴ | 2.04 × 10⁻¹ |
| rs4843865 | G | 84,526,806 | T/A | 0.17/0.21 | 2.93 × 10⁻⁸ | 0.78 (0.72-0.85) | 6.64 × 10⁻¹ | 1.46 × 10⁻² |
| rs11347703 | I | 84,527,141 | G/— | 0.18/0.21 | 1.11 × 10⁻⁸ | 0.78 (0.72-0.85) | 5.73 × 10⁻¹ | — |
| rs8052690 | G | 84,527,239 | A/G | 0.18/0.21 | 5.69 × 10⁻⁸ | 0.79 (0.72-0.86) | 6.58 × 10⁻¹ | 5.28 × 10⁻³ |
| rs186249 | G | 84,528,397 | G/C | 0.30/0.26 | 1.66 × 10⁻⁵ | 1.19 (1.10-1.28) | 1.63 × 10⁻² | 9.37 × 10⁻¹ |
| rs11117422 | G | 84,529,514 | G/C | 0.17/0.21 | 9.37 × 10⁻⁹ | 0.77 (0.71-0.84) | 6.76 × 10⁻¹ | 1.13 × 10⁻² |
| rs11644034 | G | 84,530,113 | G/A | 0.17/0.20 | 2.36 × 10⁻⁸ | 0.78 (0.71-0.85) | 5.10 × 10⁻¹ | 2.63 × 10⁻² |
| rs305066 | I | 84,530,277 | C/T | 0.33/0.29 | 8.18 × 10⁻⁶ | 1.18 (1.10-1.27) | — | 3.62 × 10⁻¹ |
| rs13335265 | G | 84,530,311 | C/G | 0.16/0.20 | 1.23 × 10⁻⁸ | 0.77 (0.70-0.84) | 2.86 × 10⁻¹ | 1.06 × 10⁻² |
| rs12711490 | G | 84,530,529 | A/G | 0.17/0.20 | 2.11 × 10⁻⁸ | 0.78 (0.71-0.85) | 4.51 × 10⁻¹ | 7.55 × 10⁻² |
| rs11641153 | I | 84,530,641 | A/G | 0.16/0.20 | 1.31 × 10⁻⁹ | 0.76 (0.70-0.83) | 4.85 × 10⁻¹ | 7.02 × 10⁻² |
| rs11641155 | I | 84,530,653 | A/G | 0.16/0.20 | 1.23 × 10⁻⁹ | 0.76 (0.70-0.83) | — | 7.02 × 10⁻² |
| rs7205434 | I | 84,530,696 | C/G | 0.16/0.20 | 1.23 × 10⁻⁹ | 0.76 (0.70-0.83) | 4.85 × 10⁻¹ | 7.02 × 10⁻² |
| rs4843868 | I | 84,530,902 | C/T | 0.16/0.20 | 8.57 × 10⁻¹⁰ | 0.76 (0.70-0.83) | 5.82 × 10⁻¹ | 7.02 × 10⁻² |
| rs305063 | G | 84,532,158 | C/A | 0.32/0.29 | 7.34 × 10⁻⁵ | 1.17 (1.08-1.26) | — | 9.66 × 10⁻¹ |
| rs4843323 | I | 84,532,462 | C/T | 0.16/0.20 | 7.71 × 10⁻¹⁰ | 0.76 (0.70-0.83) | — | — |
| rs4843869 | I | 84,532,642 | G/A | 0.16/0.20 | 7.61 × 10⁻¹⁰ | 0.76 (0.70-0.83) | 4.19 × 10⁻¹ | 6.59 × 10⁻² |
| rs7202472 | G | 84,535,003 | C/A | 0.15/0.19 | 7.25 × 10⁻⁹ | 0.77 (0.70-0.84) | 3.94 × 10⁻¹ | 1.41 × 10⁻² |
| rs11117426 | G | 84,547,768 | A/G | 0.16/0.19 | 2.42 × 10⁻⁴ | 0.84 (0.77-0.92) | 1.07 × 10⁻¹ | 2.12 × 10⁻⁵ |
| rs11117427 | G | 84,548,058 | G/A | 0.16/0.18 | 3.46 × 10⁻⁴ | 0.84 (0.77-0.93) | — | 1.99 × 10⁻⁵ |
| rs12445476 | G | 84,548,770 | A/C | 0.16/0.18 | 1.76 × 10⁻⁴ | 0.84 (0.76-0.92) | 6.49 × 10⁻² | 2.19 × 10⁻⁵ |
| rs11642873 | G | 84,549,206 | A/C | 0.15/0.18 | 2.92 × 10⁻⁴ | 0.84 (0.77-0.92) | 8.82 × 10⁻¹ | 5.63 × 10⁻⁵ |
| rs34912238 | I | 84,559,404 | C/T | 0.16/0.19 | 2.15 × 10⁻⁵ | 0.82 (0.75-0.90) | — | — |

The following abbreviations are used: G, genotyped; I, imputed; MAF, minor-allele frequency; OR, odds ratio; and CI, confidence interval.
^{a}All subjects, including the 374 that were removed so that the replication study was independent from the GWA scan, were imputed. Tables S4 and S5 contain results for all populations evaluated within this study.
^{b}Major/minor alleles.
^{c}Case/control.

TABLE 3
Replicated Loci that Demonstrate Suggestive Evidence of SLE Risk^{a}

| | | | | | | | | Test of | |
|---|---|---|---|---|---|---|---|---|---|
| | | | European | | | | | Heterogeneity | |
| SNP | Locus | Alleles^{b} | p_{GWA scan}^{c} | OR_{GWA scan} (95% CI) | p_{REP} | OR_{REP} (95% CI) | p_{META-Euro} | p [symbol] | [symbol] |
| rs7542235 | CFHR1 | A/G | 3.94 × 10⁻³ | 1.30 (1.11-1.54) | 1.10 × 10⁻³ | 1.15 (1.06-1.25) | 1.85 × 10⁻⁵ | 0.180 | 44.4% |
| rs485499 | LOC730109/IL12A | A/G | 2.14 × 10⁻³ | 0.75 (0.65-0.87) | 1.47 × 10⁻⁴ | 0.87 (0.81-0.94) | 1.31 × 10⁻⁶ | 0.076 | 68.2% |
| rs669003 | LOC730109/IL12A | A/G | 2.16 × 10⁻³ | 0.75 (0.65-0.87) | 1.15 × 10⁻⁴ | 0.87 (0.81-0.93) | 1.02 × 10⁻⁶ | 0.081 | 67.2% |
| rs7631930 | LPP | A/G | 3.60 × 10⁻³ | 1.25 (1.06-1.49) | 1.66 × 10⁻³ | 1.15 (1.05-1.25) | 2.71 × 10⁻⁵ | 0.375 | 0.00% |
| rs9310002 | CADM2 | G/A | 2.09 × 10⁻³ | 2.06 (1.38-3.07) | 6.12 × 10⁻³ | 1.39 (1.10-1.76) | 8.30 × 10⁻⁵ | 0.099 | 63.3% |
| rs1075059 | LOC63920 | A/C | 7.30 × 10⁻⁴ | 0.82 (0.71-0.95) | 7.26 × 10⁻³ | 0.91 (0.85-0.97) | 5.27 × 10⁻⁵ | 0.221 | 33.2% |
| rs1895321 | SLU7 | A/C | 4.21 × 10⁻³ | 1.22 (1.06-1.41) | 3.11 × 10⁻³ | 1.11 (1.04-1.19) | 6.09 × 10⁻⁵ | 0.260 | 21.2% |
| rs7039790 | ADAMTSL1 | C/A | 5.36 × 10⁻³ | 1.62 (1.24-2.12) | 1.14 × 10⁻³ | 1.27 (1.10-1.47) | 2.38 × 10⁻⁵ | 0.124 | 57.7% |
| rs2940712 | C10orf64 | G/A | 4.72 × 10⁻³ | 0.79 (0.67-0.91) | 8.73 × 10⁻⁴ | 0.88 (0.82-0.95) | 1.62 × 10⁻⁵ | 0.178 | 45.0% |
| rs10790605 | OR8D4 | G/A | 2.02 × 10⁻³ | 0.80 (0.67-0.95) | 4.35 × 10⁻³ | 0.88 (0.81-0.96) | 5.39 × 10⁻⁵ | 0.321 | 0.00% |
| rs7960162 | FAM19A2 | A/G | 4.95 × 10⁻³ | 0.76 (0.61-0.94) | 4.31 × 10⁻³ | 0.87 (0.79-0.96) | 9.79 × 10⁻⁵ | 0.253 | 23.6% |
| rs749373 | STXBP6 | A/G | 5.44 × 10⁻³ | 1.34 (1.11-1.62) | 2.32 × 10⁻³ | 1.16 (1.05-1.27) | 5.26 × 10⁻⁵ | 0.171 | 46.7% |

The following abbreviation is used: GWA, genome-wide association; OR, odds ratio; and CI, confidence interval; and REP, replication.
^{a}Table S7 contains results for all populations evaluated within this study.
^{b}Major/minor alleles.
^{c}GWA scan previously reported in Graham et. al. [symbol]
^{d}Cochran's Q test statistic. [symbol]
indicates data missing or illegible when filed

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### VII. REFERENCES

The following references, to the extent that they provide exemplary procedural or other details supplementary to those set forth herein, are specifically incorporated herein by reference.
U.S. Pat. No. 4,659,774
U.S. Pat. No. 4,683,195
U.S. Pat. No. 4,683,202
U.S. Pat. No. 4,800,159
U.S. Pat. No. 4,816,571
U.S. Pat. No. 5,141,813
U.S. Pat. No. 5,242,974
U.S. Pat. No. 5,264,566
U.S. Pat. No. 5,384,261
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,436,327
U.S. Pat. No. 5,445,934
U.S. Pat. No. 5,472,672
U.S. Pat. No. 5,527,681
U.S. Pat. No. 5,529,756
U.S. Pat. No. 5,532,128
U.S. Pat. No. 5,545,531
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,593,839
U.S. Pat. No. 5,599,695
U.S. Pat. No. 5,602,244
U.S. Pat. No. 5,624,711
U.S. Pat. No. 5,658,734
U.S. Pat. No. 5,700,637
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 6,004,755
EP Application No. 329 822
Frohman, In: PCR Protocols: A Guide To Methods And Applications, Academic Press, N.Y., 1990.
GB Application 2 202 328
Gillam et al., J. Biol. Chem., 253(8):2532-2539, 1978.
Guilfoyle et al., Nucleic Acids Research, 25:1854-1858, 1997.
Innis et al., Proc. Natl. Acad. Sci. USA, 85(24):9436-9440, 1988.
Itakura et al., J. Am. Chem. Soc., 97(25):7327-7332, 1975.
Khorana, Science, 203(4381):614-625, 1979.
Kwoh et al., Proc. Natl. Acad. Sci. USA, 86: 1173, 1989.
Ohara et al., Proc. Natl. Acad. Sci. USA, 86: 5673-5677, 1989.
PCT Appln. PCT/US87/00880
PCT Appln. PCT/US89/01025
PCT Appln. WO 88/10315
PCT Appln. WO 89/06700
PCT Appln. WO 89/01025
PCT Appln. WO 90/07641
Sambrook et al., In: Molecular cloning, Cold Spring Harbor Laboratory Press, Cold Spring Harbor, N.Y., 1989, 1990.
Walker et al., Proc. Natl. Acad. Sci. USA, 89:392-396 1992.
Beaucage, Methods Mol. Biol., 20:33-61,1993.
1. Petri, M. (2002). Epidemiology of systemic lupus erythematosus. Best Pract Res Clin Rheumatol 16, 847-858.
2. Moser, K. L., Kelly, J. A., Lessard, C. J., and Harley, J. B. (2009). Recent insights into the genetic basis of systemic lupus erythematosus. Genes Immun 10, 373-379.
3. Alarcon-Segovia, D., Alarcon-Riquelme, M. E., Cardiel, M. H., Caeiro, F., Massardo, L., Villa, A. R., and Pons-Estel, B. A. (2005). Familial aggregation of systemic lupus erythematosus, rheumatoid arthritis, and other autoimmune diseases in 1,177 lupus patients from the GLADEL cohort. Arthritis Rheum 52, 1138-1147.
4. Anaya, J. M., Tobon, G. J., Vega, P., and Castiblanco, J. (2006). Autoimmune disease aggregation in families with primary Sjogren's syndrome. J Rheumatol 33, 2227-2234.
5. Arora-Singh, R. K., Assassi, S., del Junco, D. J., Arnett, F. C., Perry, M., Irfan, U., Sharif, R., Mattar, T., and Mayes, M. D. (2010). Autoimmune diseases and autoantibodies in the first degree relatives of patients with systemic sclerosis. J Autoimmun 35, 52-57.
6. Sestak, A. L., Shaver, T. S., Moser, K. L., Neas, B. R., and Harley, J. B. (1999). Familial aggregation of lupus and autoimmunity in an unusual multiplex pedigree. J Rheumatol 26, 1495-1499.
7. Deng, Y., and Tsao, B. P. (2010). Genetic susceptibility to systemic lupus erythematosus in the genomic era. Nat Rev Rheumatol 6, 683-692.
8. Reinertsen, J. L., Klippel, J. H., Johnson, A. H., Steinberg, A. D., Decker, J. L., and Mann, D. L. (1978). B-lymphocyte alloantigens associated with systemic lupus erythematosus. N Engl J Med 299, 515-518.
9. Nies, K. M., Brown, J. C., Dubois, E. L., Quismorio, F. P., Friou, G. J., and Terasaki, P. I. (1974). Histocompatibility (HL-A) antigens and lymphocytotoxic antibodies in systemic lupus erythematosus (SLE). Arthritis Rheum 17, 397-402.
10. Graham, R. R., Ortmann, W. A., Langefeld, C. D., Jawaheer, D., Selby, S. A., Rodine, P. R., Baechler, E. C., Rohlf, K. E., Shark, K. B., Espe, K. J., et al. (2002). Visualizing human leukocyte antigen class II risk haplotypes in human systemic lupus erythematosus. Am J Hum Genet 71, 543-553.
11. McCulloch, D. K., Klaff, L. J., Kahn, S. E., Schoenfeld, S. L., Greenbaum, C. J., Mauseth, R. S., Benson, E. A., Nepom, G. T., Shewey, L., and Palmer, J. P. (1990). Nonprogression of subclinical β-cell dysfunction among first-degree relatives of IDDM patients. 5-yr follow-up of the Seattle Family Study. Diabetes 39, 549-556.
12. Baechler, E. C., Batliwalla, F. M., Karypis, G., Gaffney, P. M., Ortmann, W. A., Espe, K. J., Shark, K. B., Grande, W. J., Hughes, K. M., Kapur, V., et al. (2003). Interferon-inducible gene expression signature in peripheral blood cells of patients with severe lupus. Proc Natl Acad Sci USA 100, 2610-2615.
13. Bennett, L., Palucka, A. K., Arce, E., Cantrell, V., Borvak, J., Banchereau, J., and Pascual, V. (2003). Interferon and granulopoiesis signatures in systemic lupus erythematosus blood. J Exp Med 197, 711-723.
14. Kirou, K. A., Lee, C., George, S., Louca, K., Papagiannis, I. G., Peterson, M. G., Ly, N., Woodward, R. N., Fry, K. E., Lau, A. Y., et al. (2004). Coordinate overexpression of interferon-cc-induced genes in systemic lupus erythematosus. Arthritis Rheum 50, 3958-3967.
15. Sigurdsson, S., Nordmark, G., Goring, H. H., Lindroos, K., Wiman, A. C., Sturfelt, G., Jonsen, A., Rantapaa-Dahlqvist, S., Moller, B., Kere, J., et al. (2005). Polymorphisms in the tyrosine kinase 2 and interferon regulatory factor 5 genes are associated with systemic lupus erythematosus. Am J Hum Genet 76, 528-537.
16. Hom, G., Graham, R. R., Modrek, B., Taylor, K. E., Ortmann, W., Garnier, S., Lee, A. T., Chung, S. A., Ferreira, R. C., Pant, P. V., et al. (2008). Association of systemic lupus erythematosus with C8orf13-BLK and ITGAM-ITGAX. N Engl J Med 358, 900-909.
17. Harley, J. B., Alarcon-Riquelme, M. E., Criswell, L. A., Jacob, C. O., Kimberly, R. P., Moser, K. L., Tsao, B. P., Vyse, T. J., Langefeld, C. D., Nath, S. K., et al. (2008). Genome-wide association scan in women with systemic lupus erythematosus identifies susceptibility variants in ITGAM, PXK, KIAA1542 and other loci. Nat Genet 40, 204-210.
18. Yang, W., Shen, N., Ye, D. Q., Liu, Q., Zhang, Y., Qian, X. X., Hirankarn, N., Ying, D., Pan, H. F., Mok, C. C., et al. (2010). Genome-wide association study in Asian populations identifies variants in ETS1 and WDFY4 associated with systemic lupus erythematosus. PLoS Genet 6, e1000841.
19. Graham, R. R., Cotsapas, C., Davies, L., Hackett, R., Lessard, C. J., Leon, J. M., Burtt, N. P., Guiducci, C., Parkin, M., Gates, C., et al. (2008). Genetic variants near TNFAIP3 on 6q23 are associated with systemic lupus erythematosus. Nat Genet 40, 1059-1061.
20. Kozyrev, S. V., Abelson, A. K., Wojcik, J., Zaghlool, A., Ling a Reddy, M. V., Sanchez, E., Gunnarsson, I., Svenungsson, E., Sturfelt, G., Jonsen, A., et al. (2008). Functional variants in the B-cell gene BANK1 are associated with systemic lupus erythematosus. Nat Genet 40, 211-216.
21. Han, J. W., Zheng, H. F., Cui, Y., Sun, L. D., Ye, D. Q., Hu, Z., Xu, J. H., Cai, Z. M., Huang, W., Zhao, G. P., et al. (2009). Genome-wide association study in a Chinese Han population identifies nine new susceptibility loci for systemic lupus erythematosus. Nat Genet 41, 1234-1237.
22. Gateva, V., Sandling, J. K., Horn, G., Taylor, K. E., Chung, S. A., Sun, X., Ortmann, W., Kosoy, R., Ferreira, R. C., Nordmark, G., et al. (2009). A large-scale replication study identifies TNIP1, PRDM1, JAZF1, UHRF1BP1 and IL10 as risk loci for systemic lupus erythematosus. Nat Genet 41, 1228-1233.
23. Lessard, C. J., Adrianto, I., Kelly, J. A., Kaufman, K. M., Grundahl, K. M., Adler, A., Williams, A. H., Gallant, C. J., Anaya, J. M., Bae, S. C., et al. (2011). Identification of a systemic lupus erythematosus susceptibility locus at 11p13 between PDHX and CD44 in a multiethnic study. Am J Hum Genet 88, 83-91.
24. Namjou, B., Kothari, P. H., Kelly, J. A., Glenn, S. B., Ojwang, J. O., Adler, A., Alarcon-Riquelme, M. E., Gallant, C. J., Boackle, S. A., Criswell, L. A., et al. (2011). Evaluation of the TREX1 gene in a large multi-ancestral lupus cohort. Genes Immun 12, 270-279.
25. Adrianto, I., Wen, F., Templeton, A., Wiley, G., King, J. B., Lessard, C. J., Bates, J. S., Hu, Y., Kelly, J. A., Kaufman, K. M., et al. (2011). Association of a functional variant downstream of TNFAIP3 with systemic lupus erythematosus. Nat Genet 43, 253-258.
26. Tan, W., Sunahori, K., Zhao, J., Deng, Y., Kaufman, K. M., Kelly, J. A., Langefeld, C. D., Williams, A. H., Comeau, M. E., Ziegler, J. T., et al. (2011). Association of PPP2CA polymorphisms with systemic lupus erythematosus susceptibility in multiple ethnic groups. Arthritis Rheum 63, 2755-2763.
27. Zhao, J., Wu, H., Khosravi, M., Cui, H., Qian, X., Kelly, J. A., Kaufman, K. M., Langefeld, C. D., Williams, A. H., Comeau, M. E., et al. (2011). Association of genetic variants in complement factor H and factor H-related genes with systemic lupus erythematosus susceptibility. PLoS Genet 7, e1002079.
28. Sanchez, E., Nadig, A., Richardson, B. C., Freedman, B. I., Kaufman, K. M., Kelly, J. A., Niewold, T. B., Kamen, D. L., Gilkeson, G. S., Ziegler, J. T., et al. (2011). Phenotypic associations of genetic susceptibility loci in systemic lupus erythematosus. Ann Rheum Dis 70, 1752-1757.
29. Hochberg, M. C. (1997). Updating the American College of Rheumatology revised criteria for the classification of systemic lupus erythematosus. Arthritis Rheum 40, 1725.
30. Barrett, J. C., Fry, B., Maller, J., and Daly, M. J. (2005). Haploview: analysis and visualization of LD and haplotype maps. Bioinformatics 21, 263-265.
31. Price, A. L., Patterson, N. J., Plenge, R. M., Weinblatt, M. E., Shadick, N. A., and Reich, D. (2006). Principal components analysis corrects for stratification in genome-wide association studies. Nat Genet 38, 904-909.
32. McKeigue, P.M., Carpenter, J. R., Parra, E. J., and Shriver, M. D. (2000). Estimation of admixture and detection of linkage in admixed populations by a Bayesian approach: application to African-American populations. Ann Hum Genet 64, 171-186.
33. Hoggart, C. J., Parra, E. J., Shriver, M. D., Bonilla, C., Kittles, R. A., Clayton, D. G., and McKeigue, P. M. (2003). Control of confounding of genetic associations in stratified populations. Am J Hum Genet 72, 1492-1504.
34. Hoggart, C. J., Shriver, M. D., Kitties, R. A., Clayton, D. G., and McKeigue, P. M. (2004). Design and analysis of admixture mapping studies. Am J Hum Genet 74, 965-978.
35. Smith, M. W., Patterson, N., Lautenberger, J. A., Truelove, A. L., McDonald, G. J., Waliszewska, A., Kessing, B. D., Malasky, M. J., Scafe, C., Le, E., et al. (2004). A high-density admixture map for disease gene discovery in african americans. Am J Hum Genet 74, 1001-1013.
36. Halder, I., Shriver, M., Thomas, M., Fernandez, J. R., and Frudakis, T. (2008). A panel of ancestry informative markers for estimating individual biogeographical ancestry and admixture from four continents: utility and applications. Hum Mutat 29, 648-658.
37. Purcell, S., Neale, B., Todd-Brown, K., Thomas, L., Ferreira, M. A., Bender, D., Maller, J., Sklar, P., de Bakker, P. I., Daly, M. J., et al. (2007). PLINK: a tool set for whole-genome association and population-based linkage analyses. Am J Hum Genet 81, 559-575.
38. Willer, C. J., Li, Y., and Abecasis, G. R. (2010). METAL: fast and efficient meta-analysis of genomewide association scans. Bioinformatics 26, 2190-2191.
39. Cochran, W. G. (1954). The Combination of Estimates from Different Experiments. Biometrics 10, 101-129.
40. Higgins, J. P., Thompson, S. G., Deeks, J. J., and Altman, D. G. (2003). Measuring inconsistency in meta-analyses. Bmj 327, 557-560.
41. Li, H., and Durbin, R. (2009). Fast and accurate short read alignment with Burrows-Wheeler transform. Bioinformatics 25, 1754-1760.
42. McKenna, A., Hanna, M., Banks, E., Sivachenko, A., Cibulskis, K., Kernytsky, A., Garimella, K., Altshuler, D., Gabriel, S., Daly, M., et al. (2010). The Genome Analysis Toolkit: a MapReduce framework for analyzing next-generation DNA sequencing data. Genome Res 20, 1297-1303.
43. Browning, S. R., and Browning, B. L. (2007). Rapid and accurate haplotype phasing and missing-data inference for whole-genome association studies by use of localized haplotype clustering. Am J Hum Genet 81, 1084-1097.
44. Robinson, J. T., Thorvaldsdottir, H., Winckler, W., Guttman, M., Lander, E. S., Getz, G., and Mesirov, J. P. (2011). Integrative genomics viewer. Nat Biotechnol 29, 24-26.
45. Frazer, K. A., Ballinger, D. G., Cox, D. R., Hinds, D. A., Stuve, L. L., Gibbs, R. A., Belmont, J. W., Boudreau, A., Hardenbol, P., Leal, S. M., et al. (2007). A second generation human haplotype map of over 3.1 million SNPs. Nature 449, 851-861.
46. Howie, B. N., Donnelly, P., and Marchini, J. (2009). A flexible and accurate genotype imputation method for the next generation of genome-wide association studies. PLoS Genet 5, e1000529.
47. Via, M., Gignoux, C., and Burchard, E.G. (2010). The 1000 Genomes Project: new opportunities for research and social challenges. Genome Med 2, 3.
48. (2010). A map of human genome variation from population-scale sequencing. Nature 467, 1061-1073.
49. Birney, E., Stamatoyannopoulos, J. A., Dutta, A., Guigo, R., Gingeras, T. R., Margulies, E. H., Weng, Z., Snyder, M., Dermitzakis, E. T., Thurman, R. E., et al. (2007). Identification and analysis of functional elements in 1% of the human genome by the ENCODE pilot project. Nature 447, 799-816.
50. Tamura, T., Yanai, H., Savitsky, D., and Taniguchi, T. (2008). The IRF family transcription factors in immunity and oncogenesis. Annu Rev Immunol 26, 535-584.
51. Baechler, E. C., Gregersen, P. K., and Behrens, T. W. (2004). The emerging role of interferon in human systemic lupus erythematosus. Curr Opin Immunol 16, 801-807.
52. Baechler, E. C., Batliwalla, F. M., Reed, A. M., Peterson, E. J., Gaffney, P. M., Moser, K. L., Gregersen, P. K., and Behrens, T. W. (2006). Gene expression profiling in human autoimmunity. Immunol Rev 210, 120-137.
53. De Jager, P. L., Jia, X., Wang, J., de Bakker, P. I., Ottoboni, L., Aggarwal, N. T., Piccio, L., Raychaudhuri, S., Tran, D., Aubin, C., et al. (2009). Meta-analysis of genome scans and replication identify CD6, IRF8 and TNFRSF1A as new multiple sclerosis susceptibility loci. Nat Genet 41, 776-782.
54. (2010). Comprehensive follow-up of the first genome-wide association study of multiple sclerosis identifies KIF21B and TMEM39A as susceptibility loci. Hum Mol Genet 19, 953-962.
55. Franke, A., McGovern, D. P., Barrett, J. C., Wang, K., Radford-Smith, G. L., Ahmad, T., Lees, C. W., Balschun, T., Lee, J., Roberts, R., et al. (2010). Genome-wide meta-analysis increases to 71 the number of confirmed Crohn's disease susceptibility loci. Nat Genet 42, 1118-1125.
56. Liu, X., Invernizzi, P., Lu, Y., Kosoy, R., Bianchi, I., Podda, M., Xu, C., Xie, G., Macciardi, F., Selmi, C., et al. (2010). Genome-wide meta-analyses identify three loci associated with primary biliary cirrhosis. Nat Genet 42, 658-660.
57. Hirschfield, G. M., Liu, X., Han, Y., Gorlov, I. P., Lu, Y., Xu, C., Chen, W., Juran, B. D., Coltescu, C., Mason, A. L., et al. (2010). Variants at IRF5-TNPO3, 17q12-21 and MMEL1 are associated with primary biliary cirrhosis. Nat Genet 42, 655-657.
58. Stahl, E. A., Raychaudhuri, S., Remmers, E. F., Xie, G., Eyre, S., Thomson, B. P., Li, Y., Kurreeman, F. A., Zhernakova, A., Hinks, A., et al. (2010). Genome-wide association study meta-analysis identifies seven new rheumatoid arthritis risk loci. Nat Genet 42, 508-514.
59. Anderson, C. A., Boucher, G., Lees, C. W., Franke, A., D'Amato, M., Taylor, K. D., Lee, J. C., Goyette, P., Imielinski, M., Latiano, A., et al. (2011). Meta-analysis identifies 29 additional ulcerative colitis risk loci, increasing the number of confirmed associations to 47. Nat Genet 43, 246-252.
60. Hirschfield, G. M., Liu, X., Xu, C., Lu, Y., Xie, G., Gu, X., Walker, E. J., Jing, K., Juran, B. D., Mason, A. L., et al. (2009). Primary biliary cirrhosis associated with HLA, IL12A, and IL12RB2 variants. N Engl J Med 360, 2544-2555.
61. Sun, J., Matthias, G., Mihatsch, M. J., Georgopoulos, K., and Matthias, P. (2003). Lack of the transcriptional coactivator OBF-1 prevents the development of systemic lupus erythematosus-like phenotypes in Aiolos mutant mice. J Immunol 170, 1699-1706.
62. (2010). IL12A, MPHOSPH9/CDK2AP1 and RGS1 are novel multiple sclerosis susceptibility loci. Genes Immun 11, 397-405.
63. Peluso, I., Pallone, F., and Monteleone, G. (2006). Interleukin-12 and Th1 immune response in Crohn's disease: pathogenetic relevance and therapeutic implication. World J Gastroenterol 12, 5606-5610.
64. Remmers, E. F., Plenge, R. M., Lee, A. T., Graham, R. R., Hom, G., Behrens, T. W., de Bakker, P. I., Le, J. M., Lee, H. S., Batliwalla, F., et al. (2007). STAT4 and the risk of rheumatoid arthritis and systemic lupus erythematosus. N Engl J Med 357, 977-986.
65. Jin, Y., Birlea, S. A., Fain, P. R., Gowan, K., Riccardi, S. L., Holland, P. J., Mailloux, C. M., Sufit, A. J., Hutton, S. M., Amadi-Myers, A., et al. (2010). Variant of TYR and autoimmunity susceptibility loci in generalized vitiligo. N Engl J Med 362, 1686-1697.
66. Hunt, K. A., Zhernakova, A., Turner, G., Heap, G. A., Franke, L., Bruinenberg, M., Romanos, J., Dinesen, L. C., Ryan, A. W., Panesar, D., et al. (2008). Newly identified genetic risk variants for celiac disease related to the immune response. Nat Genet 40, 395-402.

What is claimed is:

1. A method of identifying a subject afflicted with or at risk of developing an Systemic Lupus Erythematosus comprising:
(a) providing a nucleic acid-containing sample from said subject;
(b) determining the presence or absence of a single nucleotide polymorphism (SNP) in Interferon regulatory factor 8 (IRF8); and
(c) identifying said subject as afflicted or at risk of development of Systemic Lupus Erythematosus when the presence of a SNP in IRF8 is observed.

2. The method of claim 1, further comprising determining the presence or absence of a second SNP from IRF8.

3. The method of claim 1, wherein the SNP is rs11644034 and/or rs11648084.

4. The method of claim 1, further comprising treating said subject based on the results of step (b).

5. The method of claim 1, further comprising taking a clinical history from said subject.

6. The method of claim 1, wherein determining comprises nucleic acid amplification.

7. The method of claim 6, wherein amplification comprises PCR.

8. The method of claim 1, wherein determining comprises primer extension.

9. The method of claim 1, wherein determining comprises restriction digestion.

10. The method of claim 1, wherein determining comprises sequencing.

11. The method of claim 1, wherein determining comprises SNP specific oligonucleotide hybridization.

12. The method of claim 1, wherein determining comprises a DNAse protection assay.

13. The method of claim 1, wherein said sample is blood, sputum, saliva, mucosal scraping or tissue biopsy.

14. The method of claim 1, wherein determining comprises assessing the presence or absence of a genetic marker that is in linkage disequilibrium with one or more of rs11644034 and/or 11648084.

15. The method of claim 1, further comprising obtaining said sample from said subject.
